(19) United States
(12) Patent Application Publication
(10) Pub. No.: US 2024/0009430 A1
(43) Pub. Date: Jan. 11, 2024

(54) GUIDEWIRE CORE HAVING NON-ROUND CROSS-SECTIONS

(71) Applicant: Abbott Cardiovascular Systems Inc., Santa Clara, CA (US)

(72) Inventor: Jonathan P. Durcan, Temecula, CA (US)

(73) Assignee: Abbott Cardiovascular Systems Inc., Santa Clara, CA (US)

(21) Appl. No.: 17/860,710

(22) Filed: Jul. 8, 2022

(51) Int. Cl. A61M 25/09

(52) U.S. Cl. CPC A61M 25/09; A61M 2025/09083

(57) ABSTRACT

An elongated guidewire for use in angioplasty and related procedures. The guidewire includes a proximal section and a distal section. The proximal section has a plurality of round cross-section segments alternating with a plurality of non-round cross-section segments. The non-round cross-section segments can include a square, rectangle, pentagon, triangle, octagon and polygon.

### BACKGROUND

This invention relates to the field of guidewires for advancing intraluminal devices such as stent delivery catheters, balloon dilatation catheters, atherectomy catheters and the like within body lumens.

In a typical peripheral vessel procedure a guidewire is percutaneously introduced into a patient's peripheral artery, e.g., femoral artery, by means of a conventional Seldinger technique and advanced therein until the distal tip of the guidewire reaches a lesion or obstruction in the femoral artery. The peripheral artery will most commonly be a femoral artery, such as a superficial femoral artery or a common femoral artery, but could also be an iliac artery, a popliteal artery, a posterior tibial artery, a peroneal artery, an anterior tibial artery, and the like. For consistency, references below will typically be made to the femoral artery. The methods comprise a series of steps which are performed endovascularly in a femoral artery, typically the superficial femoral artery including the popliteal artery which is an extension of the superficial femoral artery, as well as in one or more adjacent femoral veins including a popliteal vein which is an extension of the femoral vein. The methods comprise forming a proximal penetration from the femoral artery to an adjacent femoral vein at a location above the occlusion. A guidewire is advanced down the femoral artery, through the proximal penetration, and into the femoral vein. Typically, the guidewire will be advanced contralaterally over the iliac arch from the opposite leg of the patient. There are two basic techniques for advancing a guidewire into the desired location within the patient's coronary anatomy, the first is a preload technique which is used primarily for over-the-wire (OTW) devices and the second is a bare wire technique which is used primarily for rapid exchange type systems. With the preload technique, a guidewire is positioned within an inner lumen of an OTW device such as a dilatation catheter or stent delivery catheter with the distal tip of the guidewire just proximal to the distal tip of the catheter and then both are advanced through the guiding catheter to the distal end thereof. The guidewire is first advanced out of the distal end of the guiding catheter into the patient's peripheral vasculature until the distal end of the guidewire crosses the location where the interventional procedure is to be performed, e.g., a lesion to be dilated or a dilated region where a stent is to be deployed. The catheter, which is slidably mounted onto the guidewire, is advanced out of the guiding catheter into the patient's vasculature over the previously introduced guidewire until the operative portion of the intravascular device, e.g., the balloon of a dilatation or a stent delivery catheter, is properly positioned across the arterial location. Once the catheter is in position with the operative means located within the desired vascular location, the interventional procedure is performed. The catheter can then be removed from the patient over the guidewire. Usually, the guidewire is left in place for a period of time after the procedure is completed to ensure reaccess to the arterial location.

With the bare wire technique, the guidewire is first advanced by itself through the guiding catheter until the distal tip of the guidewire extends beyond the arterial location where the procedure is to be performed. Then a rapid exchange (RX) catheter is mounted onto the proximal portion of the guidewire which extends out of the proximal end of the guiding catheter, which is outside of the patient. The catheter is advanced over the guidewire, while the position of the guidewire is fixed, until the operative means on the RX catheter is disposed within the vascular location where the procedure is to be performed. After the procedure, the intravascular device may be withdrawn from the patient over the guidewire or the guidewire advanced further within the vasculature for an additional procedure.

Conventional guidewires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. A shapeable member, which may be the distal extremity of the core member or a separate shaping ribbon, which is secured to the distal extremity of the core member, extends through the flexible body and is secured to the distal end of the flexible body by soldering, brazing or welding which forms a rounded distal tip. Torqueing means are provided on the proximal end of the core member to rotate, and thereby steer, the guidewire while it is being advanced through a patient's vascular system.

What has been needed is a guidewire that has a proximal segment geometry that is receptive to use with a torque device so that the physician can more easily advance the guidewire through tortuous vasculature. The present invention satisfies these and other needs by providing enhanced torque response, improved tactile feel, increased pushability as the guidewire is advanced distally, and smoother tracking through the vasculature.

### SUMMARY OF THE INVENTION

In one aspect of the invention, a guidewire is advanced into a patient with the assistance of a torque device. Since commercial torque devices have some difficulty with engagement of round cross-section guidewires, the present invention provides for a guidewire having round cross-section segments alternating with non-round cross-section segments.

A guidewire has an elongated core member having a proximal segment and a distal segment. The distal segment has a distal end which typically has a helical coil mounted thereon. As the guidewire is advanced distally into a patient's vasculature, for example into the femoral arteries, for the distal 50 cm of the guidewire there is no need for the physician to use a torque device on the guidewire because there would not be any significant tortuosity. This distance, L1 in FIG. 4 (e.g., contralateral treatment of the superficial femoral artery), signifies the length of the guidewire inserted into the patient without requiring enhanced tactile or torque device engagements. The length L1 typically is approximately 50 cm, however, it can vary in the range from 36 cm to 72 cm depending on numerous factors including the height from the patient.

Again referring to FIG. 4, the length of L2 along the proximal segment represents a portion of the guidewire that is outside the patient and requires enhanced tactile and torque device engagement since length L1 is now advancing distally into more torturous vasculature. In one embodiment, a non-round cross-section segment has a distal end and a proximal end. The distal end abuts a proximal end of the distal segment of L1. The non-round cross-section segment preferably has a minimum length of 2.0 mm and up to 30 mm. In one embodiment, the non-round cross-section segment has a length in the range from 2.0 mm to 5.0 mm. In another embodiment, the non-round cross-section segment has a length in the range from 1.0 cm to 2.0 cm. The length of the non-round cross-section segment should correspond to the length of the operable portion of a torque device. One such torque device, is commercially available and marketed by Abbott Vascular Systems, Santa Clara, California. The torque device has a plurality of collets (finger like structures) that can open and close for engagement with the non-round cross-section segment. Thus, the length of the non-round cross-section segment should be long enough to accommodate and engage with the collets of the torque device. Once the torque device is attached to the non-round cross-section segment, it can be used to torque the guidewire and advance it distally further into the patient for a length of 5.0 cm to 10.0 cm.

The non-round cross-section segment alternates along the proximal segment with round cross-section segments. The round cross-section segments have a distal end and a proximal end, and have a length L3 (FIG. 4) in the range from 5.0 cm to 10.0 cm. The length L3 coincides with the typical guidewire distal advancement increments into the patient's vasculature. The non-round cross-section segment alternates with the round cross-section segment along the proximal segment of the guidewire. There is no set number of non-round cross-section segments required, but there needs to be enough to allow the physician to use the torque device several times to advance the distal end of the distal segment into position in the patient's coronary artery. For example, if the guidewire is 300 cm long, there is no need to have the non-round cross-section segments in the proximal 100 cm of the guidewire because the distal end of the distal segment will definitely be in the proper position in the patient's coronary arteries. On the other hand, if the guidewire is 190 cm long, then the non-round cross-section segments will need to be positioned along the entire length of the proximal segment.

Since the round cross-section segments have more mass and a greater diameter than the non-round cross-section segment, they provide better torque transmission, while the non-round cross-section segments provide better surface contact with the torque device.

The non-round cross-section segments include a square cross-section segment, a triangle, a pentagon, an octagon, and a rectangle. In one embodiment, the square cross-section segments are aligned out-of-phase with each other so that as the guidewire tracks around curves, the flats on the square cross-section segments are at different contact points thereby enhancing the tracking of the guidewire.

### BRIEF DESCRIPTION OF THE DRAWINGS

FIG. 1 is an elevational view of a prior art guidewire.

FIG. 2 is an elevational view of a prior art guidewire.

FIG. 3 is a transverse cross-sectional view of the prior art guidewire of FIG. 2 taken along lines 3-3.

FIG. 4 is an elevational view of the guidewire having a proximal segment with alternating round and non-round cross-section segments.

FIG. 5 is a transverse cross-section view taken along lines 5-5 depicting a non-round cross-section segment.

FIG. 6 is a transverse cross-section view taken along lines 6-6 depicting a round cross-section segment.

FIG. 7 is a transverse cross-section view taken along lines 7-7 depicting a non-round cross-section segment that is aligned out-of-phase with the non-round cross-section segment depicted in FIG. 5.

FIG. 8 is a transverse cross-section view taken along lines 8-8 depicting a round cross-section segment.

FIG. 9 is a transverse cross-section view taken along lines 9-9 depicting a non-round cross-section segment aligned out-of-phase with the non-round cross-section segment depicted in FIG. 7.

FIG. 10 is a transverse cross-section view taken along lines 10-10 depicting a round cross-section segment.

FIG. 11 is an exploded perspective view of a commercially available guidewire torque device.

FIG. 12 is a transverse cross-sectional view of a triangular cross-section segment.

FIG. 13 is a transverse cross-section view of a pentagonal cross-section segment.

FIG. 14 is a transverse cross-section view of a octagonal cross-section segment.

FIG. 15 is a transverse cross-section view of a rectangular cross-section segment.

### DETAILED DESCRIPTION OF THE PREFERRED EMBODIMENTS

### Prior Art Guidewires

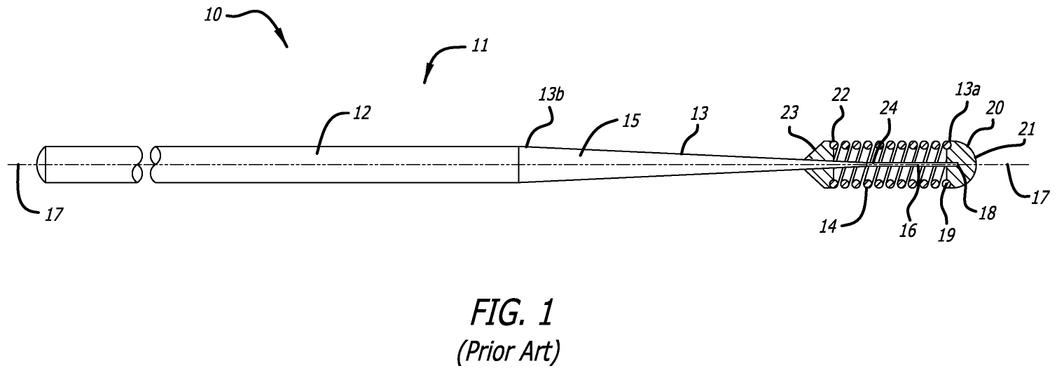

Prior art guidewires typically include an elongated core wire having a flexible atraumatic distal end. A prior art guidewire 10 is shown in FIGS. 1-3 and includes an elongated core member 11 with a proximal core section 12, a distal core section 13, and a flexible body member 14 which is fixed to the distal core section. The distal core section 13 has a tapered segment 15, a flexible segment 16 which is distally contiguous to the tapered segment 15, a distal end 13a, and a proximal end 13b. The distal section 13 may also have more than one tapered segment 15 which have typical distally decreasing tapers with substantially round transverse cross-sections. The proximal core section 12 typically has a round transverse cross-section. A longitudinal axis 17 extends the entire length of the prior art guidewire 10.

The core member 11 may be formed of stainless steel, NiTi alloys or combinations thereof. The core member 11 is optionally coated with a lubricious coating such as a fluoropolymer, e.g., TEFLON® available from DuPont, which extends the length of the proximal core section. Hydrophilic coatings may also be employed. The length and diameter of prior art guidewire 10 may be varied to suit the particular procedures in which it is to be used and the materials from which it is constructed. The length of the guidewire 10 generally ranges from about 65 cm to about 320 cm, more typically ranging from about 160 cm to about 200 cm, and preferably from about 175 cm to about 190 cm or 300 cm for the peripheral anatomy. The guidewire diameter generally ranges from about 0.008 inch to about 0.035 inch (0.203 to 0.889 mm), more typically ranging from about 0.012 inch to about 0.018 inch (0.305 to 0.547 mm), and preferably about 0.014 inch (0.336 mm) for coronary anatomy and 0.018 inch (0.547 mm) and 0.035 inch (0.889 mm) for peripheral anatomy.

The flexible segment 16 terminates in a distal end 18. Flexible body member 14, preferably a coil, surrounds a portion of the distal section of the elongated core 13, with a distal end 19 of the flexible body member 14 secured to the distal end 18 of the flexible segment 16 by the body of solder 20. The proximal end 22 of the flexible body member 14 is similarly bonded or secured to the distal core section 13 by a body of solder 23. Materials and structures other than solder may be used to join the flexible body 14 to the distal core section 13, and the term “solder body” includes other materials such as braze, epoxy, polymer adhesives, including cyanoacrylates and the like.

The wire from which the flexible body 14 is made generally has a transverse diameter of about 0.001 to about 0.004 inch, preferably about 0.002 to about 0.003 inch (0.05 mm). Multiple turns of the distal portion of the coil may be expanded to provide additional flexibility. The coil may have a diameter or transverse dimension that is about the same as the proximal core section 12. The flexible body member 14 may have a length of about 2 to about 40 cm or more, preferably about 2 to about 10 cm in length. A flexible body member 14 in the form of a coil may be formed of a suitable radiopaque material such as platinum or alloys thereof or formed of other material such as stainless steel and coated with a radiopaque material such as gold.

The flexible segment 16 has a length typically ranging about 1 to about 12 cm, preferably about 2 to about 10 cm, although longer segments may be used. The form of taper of the flexible segment 16 provides a controlled longitudinal variation and transition in flexibility (or degree of stiffness) of the core segment. The flexible segment is contiguous with the core member 11 and is distally disposed on the distal section 13 so as to serve as a shapable member.

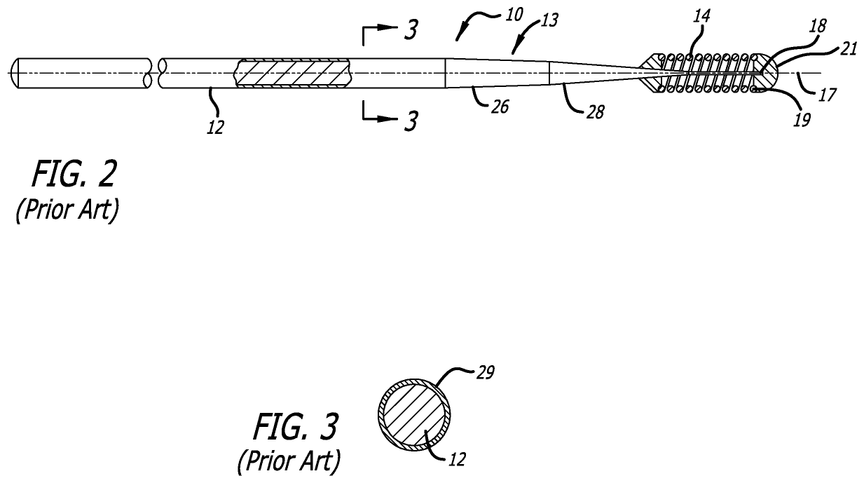

In FIGS. 2-3, the prior art guidewire 10 has a core member 32, a helical coil 14, a first tapered core segment 26, and a second tapered core segment 28, which is distally contiguous to the first tapered core segment. The second tapered core segment 28 tapers at a greater degree than the first tapered core segment and this additional taper provides a much smoother transition when the distal portion of the guidewire 10 is advanced through a tortuous passageway. The degree of taper of the first tapered core segment 26, i.e., the angle between the longitudinal axis 17 and a line tangent to the first tapered core segment 26 typically is about 0.146° , whereas the taper of the second tapered core segment 28, i.e., the angle between the longitudinal axis 17 and the second tapered core segment 28 is larger than the first angle and typically is about 0.109° such as is shown in the view of the guidewire 10 in FIG. 2. Moreover, all of the multiple tapered core segments need not have increasing degrees of tapers in the distal direction. However, two or more contiguous tapered core segments over a length of about 5 to 15 cm should have distally increasing degrees of tapering.

Typically, the first tapered core segment is about 3 cm in length and the second tapered core segment is about 4 cm in length. The guidewire 10 has a proximal segment of about 0.014 inch (0.36 mm) in diameter, the first tapered core segment has a diameter ranging from 0.014 inch down to about 0.008 inch (0.36-0.20 mm) and the second tapered core segment has a diameter ranging from about 0.008 inch to about 0.002 inch (0.20-0.05 mm). A body of solder 21 secures the proximal end of the helical coil 14 to an intermediate location on the second tapered core segment 28.

The core member 12 is coated with a lubricious coating 29 such as a fluoropolymer, e.g., TEFLON® available from DuPont, which extends the length of the proximal segment 34. The distal portion is also provided a lubricous coating, not shown for purposes of clarity, such as a MICROGLIDE™ coating used by the present assignee, Abbott Cardiovascular Systems, Inc., on many of its commercially available guidewires. A hydrophilic coating may also be employed.

The core member 12 may be formed of stainless steel, CoCr, Ti, and NiTi alloys or combinations thereof or other high strength alloys as is well known in the art.

The helical coil 14 is formed of a suitable radiopaque material such as platinum or alloys thereof or formed of other material such as stainless steel and coated with a radiopaque material such as gold. The wire from which the coil is made generally has a transverse diameter of about 0.003 inch (0.05 mm). The overall length of the helical coil 14 is typically about 3 cm. Multiple turns of the distal portion of coil 14 may be expanded to provide additional flexibility.

One of the problems associated with prior art guidewires such as shown in FIGS. 1-3, is that they have round cross-section, making it difficult for physicians to grip the guidewire and torque it and advance it in into tortuous vasculature.

### Guidewire Core Having Non-Round Cross-Section

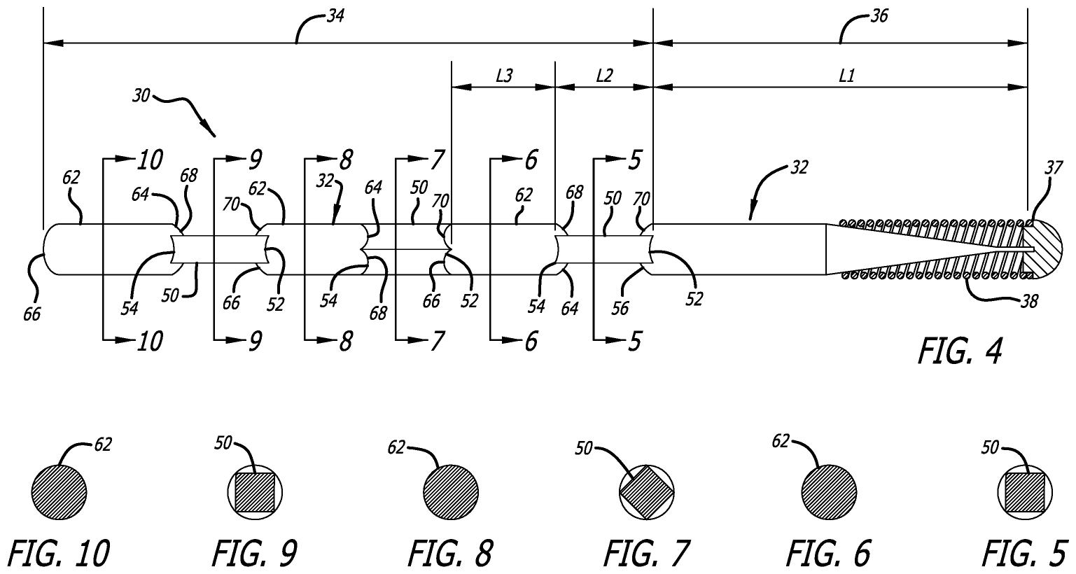

In keeping with the invention and as shown in FIGS. 4-10, a guidewire 30 has an elongated core member 32 having a proximal segment 34 and a distal segment 36. The distal segment 36 has a distal end 37 which typically has a helical coil 38 mounted thereon. As the guidewire 30 is advanced distally into a patient's vasculature, for example into the peripheral arteries, for the distal 50 cm of the guidewire 30 there is no need for the physician to use a torque device on the guidewire because there would not be any significant tortuosity. This distance, L1 in FIG. 4, signifies the length of the guidewire 30 inserted into the patient without requiring enhanced tactile or torque device engagements. The length L1 typically is approximately 50 cm, however, it can vary in the range of 36 cm to 72 cm depending on numerous factors including the height of the patient.

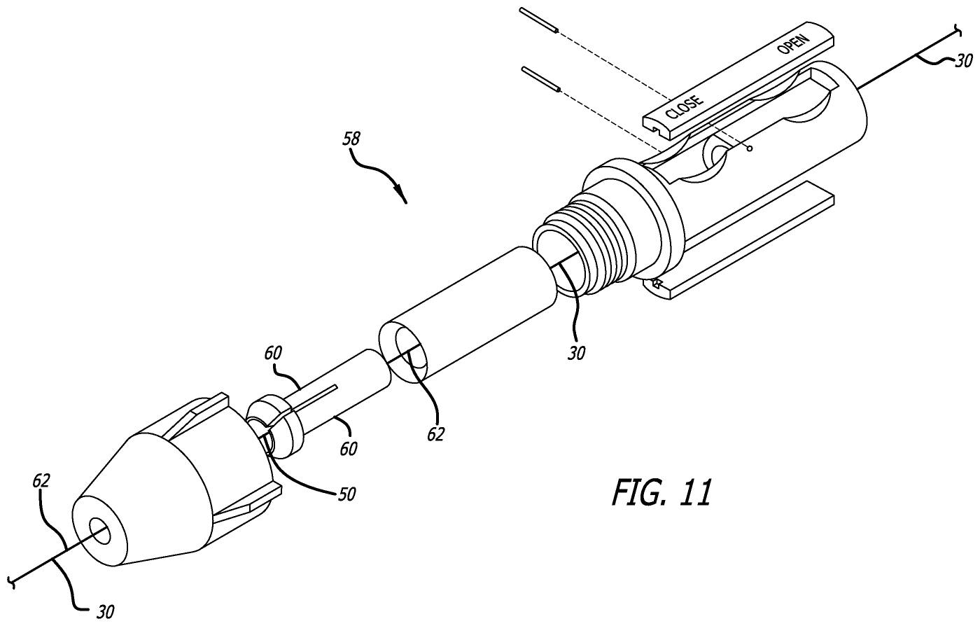

Again referring to FIG. 4, the length of L2 along the proximal segment 34 represents a portion of the guidewire 30 that is outside the patient and requires enhanced tactile and torque device engagement since length L1 is now advancing distally into more torturous vasculature. In one embodiment, as shown in FIGS. 4-10, a non-round cross-section segment 50 has a distal end 52 and a proximal end 54. The distal end 52 abuts a proximal end 56 of the distal segment 36 of L1. The non-round cross-section segment 50 preferably has a minimum length of 2.0 mm and up to 30 mm. In one embodiment, the non-round cross-section segment 50 has a length in the range from 2.0 mm to 5.0 mm. In another embodiment, the non-round cross-section segment 50 has a length in the range from 1.0 cm to 2.0 cm. The length of the non-round cross-section segment 50 should correspond to the length of the operable portion of a torque device 58. One torque device 58, shown in FIG. 11, is commercially available and has a plurality of collets 60 (finger like structures) that can open and close for engagement with the non-round cross-section segment 50. Thus, the length of the non-round cross-section segment 50 should be long enough to accommodate and engage with the collets 60 of the torque device 58. One torque device 58 is attached to the non-round cross-section segment 50, it can be used to torque the guidewire 30 and advance it distally further into the patient for a length of 5.0 cm to 10.0 cm.

The non-round cross-section segment 50 alternates along the proximal segment 34 with round cross-section segments 62. The round cross-section segments 62 have a distal end 64 and a proximal end 66, and have a length L3 in the range from 5.0 cm to 10.0 cm. The length L3 coincides with the typical guidewire 30 distal advancement increments into the patient's vasculature. As can be seen in FIGS. 4-10, the non-round cross-section segment 50 alternates with the round cross-section segment 62 along the proximal segment 34 of the guidewire 30. There is no set number of non-round cross-section segments 50 required, but there needs to be enough to allow the physician to use the torque device several times to advance the distal end 37 of the distal segment 36 into position in the patient's coronary artery. For example, if the guidewire 30 is 300 cm long, there is no need to have the non-round cross-section segments 50 in the proximal 100 cm of the guidewire 30 because the distal end 37 of the distal segment 36 will definitely be in the proper position in the patient's coronary arteries. On the other hand, if the guidewire 30 is 190 cm long, then the non-round cross-section segments will need to be positioned along the entire length of the proximal segment 34.

Since the round cross-section segments 62 have more mass and a greater diameter than the non-round cross-section segment 50, they provide better torque transmission, while the non-round cross-section segments 50 provide better surface contact with the torque device 58.

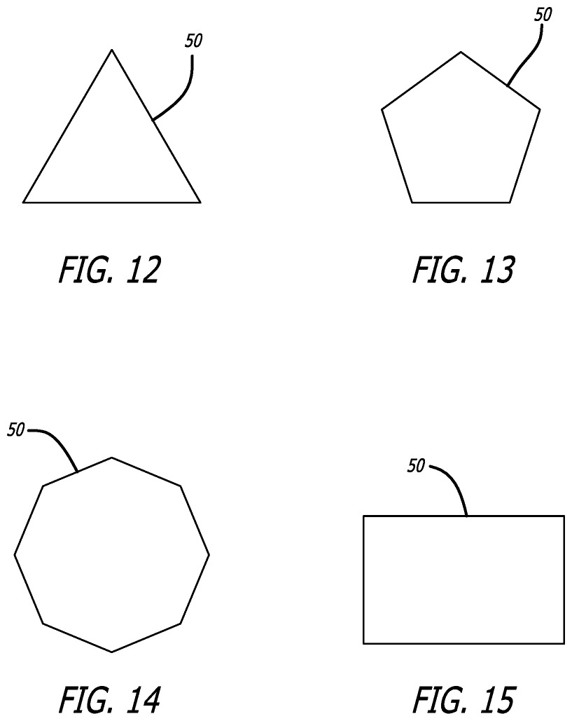

The non-round cross-section segments 50 are shown in FIGS. 5, 7, 9 and 12-15. In FIGS. 5, 7 and 9, a square cross-section segment is depicted, and in FIG. 12 a triangle, FIG. 13 a pentagon, FIG. 14 an octagon, and FIG. 15 a rectangle. In one embodiment, shown in FIGS. 5, 7 and 9, the square cross-section segments 50 are aligned out-of-phase with each other so that as the guidewire 30 tracks around curves, the flats on the square cross-section segments 50 are at different contact points thereby enhancing the tracking of the guidewire 30. The other non-round cross-section segments 50 shown in FIGS. 12-15 also can be aligned out-of-phase to provide the same results. In another embodiment, the non-round cross-section segments 50 are aligned in-phase.

In one embodiment, the guidewire 30 is formed from one continuous core wire that includes the round cross-section segments 62 and the non-round cross-section segments 50. The non-round cross-section segments 50 can be formed by multiple different processes such as grinding, laser cutting, or chemical removal, all of which is known in the art.

In one embodiment, there are rounded edges 68 and 70 where the round cross-section segments 62 abut the non-round cross-section segments 50. The rounded edges provide a smooth transition and improve guidewire 30 tracking.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those having ordinary of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Thus, it is intended that the scope of the invention herein disclosed should not be limited by the particular embodiments described above.

I claim:

1. A guidewire, comprising:
an elongated core member having a proximal segment and a distal segment, the distal segment having a round cross-section segment; and
the proximal segment having a plurality of round cross-section segments alternating with a plurality of non-round cross-section segments.

2. The guidewire of claim 1, wherein the plurality of non-round cross-section segments are taken from the group consisting of square, rectangle, pentagon, triangle, octagon and polygon.

3. The guidewire of claim 2, wherein each of the plurality of the non-round cross-section segments has a length in a range from 2.0 mm to 30.0 mm.

4. The guidewire of claim 1, wherein each of the plurality of the non-round cross-section segments has a length in a range from 2.0 mm to 5.0 mm.

5. The guidewire of claim 1, wherein each of the plurality of the round cross-section segments in the proximal segment has a length in a range from 5.0 cm to 10.0 cm.

6. The guidewire of claim 1, wherein each of the plurality of the non-round cross-section segments is a square cross-section shape.

7. The guidewire of claim 1, wherein at least one of the plurality of the non-round cross-section segments is a square cross-section segment.

8. The guidewire of claim 1, wherein the plurality of the non-round cross-section segments are aligned out-of-phase.

9. A guidewire, comprising:
an elongated core member having a proximal segment and a distal segment, the distal segment having a round cross-section segment; and
the proximal segment having at least one round cross-section segment alternating with at least one non-round cross-section segment.

10. The guidewire of claim 9, wherein the at least one non-round cross-section segment is/are taken from the group consisting of square, rectangle, pentagon, triangle, octagon and polygon.

11. The guidewire of claim 10, wherein the at least one non-round cross-section segment has a length in a range from 2.0 mm to 30.0 mm.

12. The guidewire of claim 10, wherein the at least one non-round cross-section segment has a length in a range from 2.0 mm to 5.0 mm.

13. The guidewire of claim 9, wherein the at least one round cross-section segment in the proximal segment has a length in a range from 5.0 cm to 10.0 cm.

14. The guidewire of claim 9, wherein the at least one non-round cross-segment is a square cross-section shape.

15. A guidewire, comprising:
an elongated core member having a proximal segment and a distal segment, the distal segment having a round cross-section segment; and
the proximal segment having a plurality of round cross-section segments alternating with a plurality of square cross-section segments.

16. The guidewire of claim 15, wherein each of the plurality of square cross-section segments has a length in a range from 2.0 mm to 30.0 mm.

17. The guidewire of claim 15, wherein each of the plurality of square cross-section segments has a length in a range from 2.0 mm to 5.0 mm.

18. The guidewire of claim 15, wherein each of the plurality of the round cross-section segments in the proximal segment has a length in a range from 5.0 cm to 10.0 cm.

19. The guidewire of claim 15, wherein the plurality of square cross-section segments are aligned out-of-phase.

20. The guidewire of claim 15, wherein the plurality of square cross-section segments are aligned in-phase.
